(19) United States
(12) Patent Application Publication
(10) Pub. No.: US 2021/0041449 A1
(43) Pub. Date: Feb. 11, 2021

(54) Methods for Measuring Relative Oxidation Levels of a Protein

(71) Applicant: Two-Tag Holdings Pty Ltd, West Perth, Western Australia (AU)

(72) Inventors: Pearl Lin Tan, Queens Park, Western Australia (AU); Peter Graeme Arthur, Wembley, Western Australia (AU); Zi Xiang Lim, Singapore (SG)

(21) Appl. No.: 17/041,551

(22) PCT Filed: Mar. 26, 2019
(86) PCT No.: PCT/AU2019/050267

§ 371 Date: Sep. 25, 2020

(30) Foreign Application Priority Data
Mar. 28, 2018 (AU) 2018901026

(51) Int. Cl. G01N 33/68

(52) U.S. Cl. CPC G01N 33/6842; G01N 2800/7009; G01N 2800/52

(57) ABSTRACT

A method for assessing the oxidation states of a protein in a sample, the method comprising the steps of contacting the sample with a first label adapted to selectively bind to at least one reduced cysteine group of the protein therein to form a first labelled sample; forming a sub-sample of the first labelled sample; treating the sub-sample to selectively reduce at least one reversibly oxidised cysteine group of the protein therein to form a treated sub-sample; contacting the treated sub-sample with a second label adapted to selectively bind to a reduced cysteine group of the protein to form a second labelled sample; and assessing the first and second labelled samples for a plurality of oxidation states of the protein.

### FIELD OF THE INVENTION

The present invention relates to a method for assessing the oxidation states of a protein in a sample. The invention also relates to a method for detecting protein oxidation, particularly modification caused by reactive oxygen species (ROS) and to kits and other uses of the methods described herein.

### BACKGROUND TO THE INVENTION

In humans, disease and physiological perturbations (e.g. hypoxia, heat, exercise, nutrition) can result in the generation of reactive oxygen species (ROS) which affect cellular function. How ROS affect cellular function depends on their type (e.g. hydroxyl radicals) and cellular location. For example, hydroxyl radicals generated in membranes can initiate peroxidation of lipids and, if severe enough, the resultant membrane leakiness can cause cell necrosis.

As a consequence of interest in the biological effects of ROS, biomarkers have been identified in blood and urine. For example, plasma F₂-isoprostanes, a commonly used biomarker of oxidative stress, are lipid degradation products resulting from the actions of highly ROS such as hydroxyl radicals.

Proteins are also targets of highly ROS, such as hydroxyl radicals, which can irreversibly damage proteins with deleterious consequences on protein function. A commonly used plasma assay to detect this type of protein oxidation is the protein carbonyl assay. Carbonyl derivatives are formed directly by ROS, such as hydroxyl radicals, or indirectly by secondary reactions with reactive carbonyl derivatives on carbohydrates.

In addition to irreversible oxidative damage, protein function can be affected by the oxidation of thiol groups of cysteine residues. Oxidation of thiol groups has been shown to affect the function of multiple proteins and has been linked to effects on a range of cellular pathways including proliferation, differentiation, necrosis and contractility. Thiol groups can be oxidised by milder oxidants such as hydrogen peroxide and are also particularly susceptible to oxidation by hypochlorous acid, a ROS produced during inflammatory responses. Accordingly, plasma proteins containing thiol groups are potential biomarkers for protein thiol oxidation. For example, although most thiol groups in plasma proteins are oxidised, the thiol group of cysteine 34 in human serum albumin, is only partially oxidised.

HPLC has been used to separate albumin into three forms based on the oxidation of cys34: a reduced state (—SH); a (reversibly) oxidised state which can convert back to the reduced state (—SOH, —SSX, where X is predominantly cysteine, homocysteine or glutathione); and a biologically irreversible oxidised state (—SO₂H, —SO₃H). Using HPLC, oxidation of cys34 has been shown to be increased after exercise, aging, haemodialysis patients, chronic kidney disease, diabetes, sleep apnoea and liver cirrhosis.

Although the oxidation state of cys34 appears to be useful for tracking oxidative stress in plasma, the HPLC technique is not widely used. One possible reason for the lack of widespread acceptance is that the analysis requires access to expensive HPLC equipment and associated analytical skills.

It is against this background and the problems and difficulties associated therewith that the present invention has been developed.

### SUMMARY OF THE INVENTION

According to a first aspect the present invention provides a method for assessing the oxidation states of a protein in a sample, the method comprising the steps of:
(a) contacting the sample with a first label adapted to selectively bind to at least one reduced cysteine group of the protein therein to form a first labelled sample;
(b) forming a sub-sample of the first labelled sample;
(c) treating the sub-sample to selectively reduce at least one reversibly oxidised cysteine group of the protein therein to form a treated sub-sample;
(d) contacting the treated sub-sample with a second label adapted to selectively bind to a reduced cysteine group of the protein therein formed during step (c) to form a second labelled sample; and
(e) assessing the first and second labelled samples for a plurality of oxidation states of the protein.

According to another aspect, the present invention also provides a means for monitoring the effects of ROS on the oxidation states of a protein in a sample exposed to the ROS, the method comprising the steps of:
(a) contacting the sample with a first label adapted to selectively bind to at least one reduced cysteine group of the protein therein to form a first labelled sample;
(b) forming a sub-sample of the first labelled sample;
(c) treating the sub-sample to selectively reduce at least one reversibly oxidised cysteine group of the protein therein to form a treated sub-sample;
(d) contacting the treated sub-sample with a second label adapted to selectively bind to a reduced cysteine group of the protein therein formed during step (c) to form a second labelled sample;
(e) assessing the first and second labelled samples for a plurality of oxidation states of the protein; and
(f) correlating the assessment from step (e) with the effects of the ROS.

According to another aspect, the present invention also provides a method for assessing an ROS associated pathology in a subject, the method comprising the steps of:
(a) contacting a sample from the subject with a first label adapted to selectively bind to at least one reduced cysteine group of the protein therein to form a first labelled sample;
(b) forming a sub-sample of the first labelled sample;
(c) treating the sub-sample to selectively reduce at least one reversibly oxidised cysteine group of the protein therein to form a treated sub-sample;
(d) contacting the treated sub-sample with a second label adapted to selectively bind to a reduced cysteine group of the protein therein formed during step (c) to form a second labelled sample;
(e) assessing the first and second labelled samples for a plurality of oxidation states of the protein; and
(f) correlating the assessment from step (e) with the ROS associated pathology.

According to another aspect, the present invention also provides a method for assessing the efficacy of a therapeutic intervention for a ROS associated pathology in a subject, the method comprising the steps of:
(a) contacting a sample from the subject with a first label adapted to selectively bind to at least one reduced cysteine group of the protein therein to form a first labelled sample;
(b) forming a sub-sample of the first labelled sample;
(c) treating the sub-sample to selectively reduce at least one reversibly oxidised cysteine group of the protein therein to form a treated sub-sample;
(d) contacting the treated sub-sample with a second label adapted to selectively bind to a reduced cysteine group of the protein therein formed during step (c) to form a second labelled sample;
(e) assessing the first and second labelled samples for a plurality of oxidation states of the protein; and
(f) correlating the assessment from step (e) with the ROS associated pathology in the presence and absence of the intervention.

According to another aspect, the present invention also provides a kit for assessing the oxidation states of a protein in a sample, the kit comprising:
(a) a first label adapted to selectively bind to at least one reduced cysteine group of the protein in the sample; and
(b) a reagent for selectively reducing at least one reversibly oxidised cysteine group of the protein in the sample.

### BRIEF DESCRIPTION OF THE DRAWINGS

FIG. 1 is a diagrammatic representation of the malpeg labelling technique. Ai. Available thiols (—S—H) in the plasma sample are initially trapped with malpeg. Aii. The sample is split in two, with reversibly oxidised thiols (—S—S—X) in the second sample converted to reduced thiols via thiol-disulphide exchange reactions. Aiii. Reduced thiols are labelled with malpeg. B. Following electrophoresis, albumin bound to malpeg is separated by about 5000 Da from unbound albumin. For sample 1, band A represents RA, whereas as band B represents OAR and OAI. For sample 2, band C represents RA and OAR, whereas band D represents OAI.

FIG. 2 shows the separation of differently oxidised forms of albumin using malpeg. Sample 1. Plasma incubated with malpeg as described in methods (procedure 1). Sample 2. Plasma incubated with malpeg following treatment with cysteine as described in methods (procedure 2). Sample 3. Plasma not incubated with malpeg. Sample 4. Commercial human albumin not incubated with malpeg. Sample 5 Commercial human albumin incubated with malpeg as described in methods (procedure 1). Composition of bands: A, RA; B, OA_{R} and OAᵢ; C, RA and OA_{R}; D, OAᵢ; E, RA, OA_{R} and OAᵢ; F, RA, OA_{R} and OAᵢ; G, RA and OA_{R}; H, OAᵢ.

FIG. 3 is an image of a gel showing the shift in the albumin band following incubation of plasma with thiol/disulfide exchange or reducing agents. Albumin was incubated for 30 minutes with concentrations of 10 mM for cysteine (lane 1), glutathione (lane 2), N-acetylcysteine (lane 3), mercaptoethanol (lane 4), DTT (lane 5) and TCEP (lane 6). Following incubation, 12.5 mM malpeg was added for 15 minutes.

FIG. 4 illustrates the use of fluorescent analysis to quantify albumin. Human albumin was loaded on to a gel and proteins were imaged fluorescently (A), quantified (B) and a standard curve was generated (C). The signal profile for the fluorescently imaged gel is shown below the gel image. Bi show albumin in the absence of malpeg, Bii show albumin bound to malpeg, Biii show unlabeled albumin.

FIG. 5 is a graph showing the impact of sample preparation and collection—the level of albumin oxidation following treatment with malpeg immediately after sampling blood (BI), immediately after preparation of plasma (PI), immediately after thawing frozen plasma (Th), and after 2.5 hours at room temperature (RT). * represents significantly different from immediately after. Values are expressed in mean±SE. (n=3).

FIG. 6 is a series of graphs showing the effect of treatment with hydrogen peroxide or hypochlorous acid on protein oxidation. Plasma samples were untreated (U), treated with 0.5 mM (H0.5) or 5 mM (H5) hydrogen peroxide, 0.5 mM (C0.5) or 5 mM (C5) hypochlorous acid. Levels of (A) total albumin thiol oxidation, (B) protein carbonyl in arbitrary units (au), (C) reversibly oxidised albumin and (D) irreversibly oxidised albumin are shown. * represents significantly different from untreated. # represents significantly different from equivalent concentration of hydrogen peroxide. (n=3-4); and

FIG. 7 is a series of graphs showing the effect of exercise on albumin oxidation. Capillary blood samples were collected prior to exercise (Pre), and after exercise to \dot{V}O_{2Peak}. Levels of (A) total oxidised albumin, (B) reversibly oxidised and (C) irreversibly oxidised albumin. * represents significantly different from pre-exercise value. Values are expressed in mean±SE. (n=6)

FIG. 8 is a series of graphs. FIG. 8A showing (i) total albumin (A) and other blood proteins (B) in an untreated sample; FIG. 8B showing (ii) oxidised albumin (C) and reduced albumin (D) in a sample treated with malpeg; and FIG. 8C showing irreversibly oxidised albumin (E) and reversibly oxidised and reduced albumin (F) in a sample treated with malpgeg and reduced with cysteine. The samples were processed using capillary electrophoresis in accordance with Example 4.

FIG. 9 is a graph showing the effect of moderate and high-density exercise on albumin oxidation. Finger prick blood samples were collected on a dried blood spot card embedded with malpeg, prior to and after both moderate and high-density exercise. The graph shows the percentage of reversibly oxidised albumin in the samples.

FIG. 10 is a graph showing the effect of an inflammatory skin treatment on albumin oxidation. Finger prick blood samples were collected on a dried blood spot card embedded with malpeg, prior to and after treatment. The graph shows the percentage of reversibly oxidised albumin in the samples.

FIG. 11 is a graph showing the effect of muscle damage on albumin oxidation in an untrained individual. Finger prick blood samples were collected on a dried blood spot card embedded with malpeg, prior to and for 4 days post weight training. The graph shows the percentage of reversibly oxidised albumin in the samples.

FIG. 12 is a graph showing the effect of exercise on both irreversible and reversible albumin oxidation in a patient after both moderate and high intensity exercise over two four-day exercise periods. Finger prick blood samples were collected on a dried blood spot card embedded with malpeg, prior to and after both moderate and high-density exercise periods.

FIG. 13 is a graph showing the effect of sickness on albumin oxidation in a patient. Finger prick blood samples were collected on a dried blood spot card embedded with malpeg over a period of 9 days.

FIG. 14 is a graph showing the effect of aerobic exercise on reversible albumin oxidation in two patients with different aerobic fitness levels. Finger prick blood samples were collected on a dried blood spot card embedded with malpeg, prior to and after exercise.

FIG. 15 is a graph showing changes in reversible albumin oxidation levels in a patient with a grade 1 calf muscle injury over time. Finger prick blood samples were collected on a dried blood spot card embedded with malpeg, over a period of 10 days post muscle injury.

FIG. 16 is a graph showing the effect of isometric contraction on protein oxidation level in blood of individuals with CFS (n=12) and healthy sedentary individuals (n=12.

### DETAILED DESCRIPTION OF THE INVENTION

According to a first aspect the present invention provides a method for assessing the oxidation states of a protein in a sample, the method comprising the steps of:
(a) contacting the sample with a first label adapted to selectively bind at least one reduced cysteine group of the protein therein to form a first labelled sample;
(b) forming a sub-sample of the first labelled sample;
(c) treating the sub-sample to selectively reduce at least one reversibly oxidised cysteine group of the protein therein to form a treated sub-sample;
(d) contacting the treated sub-sample with a second label adapted to selectively bind to a reduced cysteine group of the protein therein formed during step (c) to form a second labelled sample; and
(e) assessing the first and second labelled samples for a plurality of oxidation states of the protein.

Preferably, the oxidation states comprise a reversibly oxidised form.

Preferably, the oxidation states comprise an irreversibly oxidised form.

Preferably, the oxidation states comprise a reversibly and an irreversibly oxidised form.

Preferably, the protein is a protein selected from the list comprising: albumin, alpha-2-macroglobulin, fibrinogen beta chain, haptoglobin, immunoglobulin lambda constant 2, inter-alpha-trypsin inhibitor heavy chain H2, serotransferrin, immunoglobulin gamma-1 heavy chain, fibrinogen gamma chain and transthyretin.

When the protein is albumin and the oxidation state comprises a reversibly oxidised form, the reversibly oxidised form preferably comprises a reversibly oxidised cysteine group at cys34.

Preferably, the protein is an animal protein such as a fish or mammalian protein. Even more preferably, the protein is a human protein.

Preferably, the sample is a body fluid sample such as a mammalian, preferably human, body fluid sample. More preferably, the sample is selected from the list of samples comprising: blood, blood plasma, blood serum, urine, milk and saliva. The sample may also be a cell extract or some other preparation derived from biological material such as a tissue sample or extract thereof. The sample can also be part of a cell such as a sample containing mitochondria or another subcellular organelle.

The sample may also comprise a single protein or a plurality of proteins. When the sample comprises a plurality of proteins the method of the present invention can be used to assess the oxidation states of the plurality of proteins in the sample. For example, the method can be used to produce a profile that indicates which proteins in a sample have been oxidised and which ones have not.

Preferably, the first label is further adapted to trap the reduced cysteine group such that the bond formed between the label and the reduced cysteine group cannot be cleaved with a reducing agent.

When the first label is adapted to trap the reduced cysteine group it is preferably contacted with the sample as soon as possible after the sample is taken. For example, the first label may be contacted with the sample less than 1, 2, 3, 4 or 5 minutes of the sample being taken. In this regard, applicant has surprisingly discovered that the handling of a sample prior to assessing the oxidation states of proteins therein can impact on the accuracy of any assessment of oxidation states.

Preferably, the first label comprises a sulfhydryl-reactive chemical group. Even more preferably, the first label comprises a maleimide group; a haloacetyl group, such as an iodoacetyl or a bromoacetyl group; and/or a pyridyl disulphide group.

The first label may be selected from the group consisting of: maleimide, phenylmercury, iodoacetamide, vinylpyridine, methyl bromide or iodoacetate or derivatives thereof.

Preferably, this component is iodoacetamide or maleimide or a derivative thereof.

Preferably, the first label is used at a concentration of at least 3 mM, 3.6 mM, 5 mM, 6 mM, 6.25 mM, 7 mM, 8 mM, 9 mM or 10 mM for at least 5, 10, 15 or 20 minutes when contacted with the sample.

Preferably, the first label further comprises a separation member adapted to facilitate separation of a labelled compound relative to unlabeled compounds.

The separation member may be a compound with a defined molecular weight that facilitates separation based on weight differences. Even more preferably, the separation member is a polymer such as polyethylene glycol. Thus, for example, the first agent may be pegylated.

The separation member may also be a fluorescent compound capable of being imaged.

The first label may also be a mass tag or label that facilitates identification via mass spectrometry or another similar methodology. Examples of suitable mass tags include: biotin-maleimide, iodoacetamide and N-Ethylmaleimide. The first label may also be an antigen.

Preferably, the sub-sample comprises a volume of about 50% of the volume of the labelled sample.

Preferably, treating the sub-sample to selectively reduce at least one reversibly oxidised cysteine group of the protein therein comprises the step of contacting the sub-sample with an effective amount of a thiol containing agent. Preferably, the thiol containing agent is adapted to react with the reversibly oxidised cysteine group in a thiol-disulphide exchange reaction that only slightly or moderately favours reduction of the reversibly oxidised cysteine group. In this regard, the thiol containing agent may be adapted to react with the reversibly oxidised cysteine group in a reaction with an equilibrium constant (pKa) value of: less than 8 or 9 and more preferably less than 4, 5, 6, or 7.

Preferably, the thiol containing agent comprises a compound including a single thiol group. Preferably, the thiol containing agent is selected from the group comprising: cysteine, glutathione (reduced), mercaptoethanol, cysteamine, penicillamine and N-acetylcysteine.

For the purposes of the present invention, the term “selectively” when used in the phrase “selectively reduce” means that reversibly oxidised cysteine groups are reduced preferentially to any irreversibly oxidised cysteine groups in the sample. Preferably, the term “selectively reduce” means that there is no measurable reduction of any irreversibly oxidised cysteine groups in the sample. In one particular form of the invention the term “selectively reduce” means that only a subset of reversibly oxidised cysteine groups is reduced e.g. for albumin, cysteine residue 34 is selectively reduced relative to other reversibly oxidised cysteine groups in albumin.

Preferably, the thiol containing agent is used at a final concentration of at least 2 mM, 4 mM, 6 mM, 8 mM, 10 mM, 12 mM, 12.5 mM, 15 mM or 20 mM.

Preferably, the thiol containing agent is contacted with the subsample for at least 5, 10, 15, 20 or 30 minutes.

Preferably, the second label is further adapted to trap the reduced cysteine group such that the bond formed there between cannot be cleaved with a reducing agent.

Preferably, the second label comprises a sulfhydryl-reactive chemical group. Even more preferably, the second label comprises a maleimide group; a haloacetyl group, such as an iodoacetyl or a bromoacetyl group; and/or a pyridyl disulphide group.

Preferably, the second label further comprises a separation member as described herein in relation to the first label.

Preferably, the second label has the same reaction chemistry/binding characteristics as the first label.

Preferably, the second label is distinguishable from the first label. For example, the second label may incorporate a different antigen, mass, absorbance or fluorescent tag.

Preferably, the second label is used at a concentration that is higher than that used for the first label. Even more preferably, the concentration of the second label may be at least 5 mM, 7.5 mM, 10 mM, 12.5 mM or 15 mM for at least 5, 10, 15 or 20 minutes when contacted with the treated sub-sample.

The step of assessing the first and second labelled samples for a plurality of oxidation states of the protein will vary depending at least in part on the choice of the first and second label. Preferably, the step of assessing comprises applying the first and second labelled samples to size based separation such as electrophoresis.

Preferably, the method of the present invention is quantitative. Thus, the method may further comprise the step of quantifying the amount of the identified oxidation states of the protein.

Preferably, the oxidation states are quantified in relative terms.

Preferably, the oxidation states are quantified as a percentage, such as a percentage of the total amount of the protein in the sample. Preferably, the oxidation states are quantified as a percentage of the protein that is oxidised.

Preferably, the oxidation states are quantified by reference to the intensity of a signal from the first or second label.

One particularly useful means for assessing the first and second labelled samples is gel electrophoresis such as PAGE as the protein sample can be applied to PAGE and then the signals from the labels measured at particular protein bands on the gel. Another suitable technique for assessing the first and second labelled samples is capillary electrophoresis, a high-speed protein analysis technique which uses the same principle of separation as PAGE electrophoresis but is performed in a gel or polymer filled capillary tube. Dependent on the type of label, other visualising means include immunoblotting, phospho-imaging or lumi-imaging.

Alternate techniques to PAGE are immunoprecipitation or lateral flow strips (where a single protein of interest is isolated), protein or antibody arrays (where a multitude of proteins are isolated on a protein chip), and mass spectrometry and/or chromatography, where single or total protein extracts are analysed (for example by multidimensional chromatography). Mass spectrometry and the protein or antibody arrays offer the opportunity to scan 10, 100 or even 1000s of proteins very rapidly very much like microarrays.

The present invention provides a means for monitoring the effects of reactive oxygen species (ROS) on the oxidation states of a protein in a sample exposed to the ROS, the method comprising the steps of:
(a) contacting the sample with a first label adapted to selectively bind to at least onereduced cysteine group of the protein therein to form a first labelled sample;
(b) forming a sub-sample of the first labelled sample;
(c) treating the sub-sample to selectively reduce at least one reversibly oxidised cysteine group of the protein therein to form a treated sub-sample;
(d) contacting the treated sub-sample with a second label adapted to selectively bind to a reduced cysteine group of the protein therein formed during step (c) to form a second labelled sample;
(e) assessing the first and second labelled samples for a plurality of oxidation states of the protein; and
(f) correlating the assessment from step (e) with the effects of the ROS.

The ROS may be any reactive oxygen molecule capable of modifying aspects of normal cellular functioning. Preferably, the ROS is selected from the group comprising: superoxide, hydroxyl radical, peroxyl radical, alkoxyl radical, hydroperoxyl radical, hypochlorous acid, hydrogen peroxide, nitric oxide, taurine chloramine, hypobromous acid, ozone, singlet oxygen and peroxinitrite.

Many important pathologies such as stroke, heart attack and age-related degeneration are associated with ROS production. Thus, the present invention also provides a method for assessing an ROS associated pathology in a subject, the method comprising the steps of:
(a) contacting a sample from the subject with a first label adapted to selectively bind to at least one reduced cysteine group of the protein therein to form a first labelled sample;
(b) forming a sub-sample of the first labelled sample;
(c) treating the sub-sample to selectively reduce at least one reversibly oxidised cysteine group of the protein therein to form a treated sub-sample;
(d) contacting the treated sub-sample with a second label adapted to selectively bind to a reduced cysteine group of the protein therein formed during step € to form a second labelled sample;
(e) assessing the first and second labelled samples for a plurality of oxidation states of the protein; and
(f) correlating the assessment from step (e) with the ROS associated pathology.

The ROS associated pathology may be selected from the group comprising: stroke, heart attack and age-related degeneration or a disease selected from the list comprising: atherosclerosis, peripheral vascular occlusive disease, hypertension, liver disease, alcoholic liver disease, kidney disease, Crohn's disease, angina, emphysema & bronchitis, chronic obstructive lung disease, diabetes, cancer, organ transplantation such as liver transplantation related disease, coronary heart disease/heart failure, stroke/neurotrauma, cardiovascular disease, coronary obstructive pulmonary disease, high blood pressure, hypoxia, fetal distress syndrome, dystrophy, rheumatoid arthritis, amyotrophic lateral sclerosis, cystic fibrosis, sepsis (including severe sepsis), acute respiratory distress syndrome, sleep apnoea, obesity, osteoperosis, human immunodeficiency virus (HW), acquired immune deficiency syndrome (AIDS), chronic fatigue syndrome, muscle injury, concussion and neurodegenerative diseases including, but not limited to, Alzheimer's Disease and Parkinson's Disease.

The method of the present invention could also be used to assess the effects of therapeutic interventions for ROS associated pathologies. Thus, the present invention also provides a method for assessing the efficacy of a therapeutic intervention for a ROS associated pathology in a subject, the method comprising the steps of:
(a) contacting a sample from the subject with a first label adapted to selectively bind to at least one reduced cysteine group of the protein therein to form a first labelled sample;
(b) forming a sub-sample of the first labelled sample;
(c) treating the sub-sample to selectively reduce at least one reversibly oxidised cysteine group of the protein therein to form a treated sub-sample;
(d) contacting the treated sub-sample with a second label adapted to selectively bind to a reduced cysteine group of the protein therein formed during step (c) to form a second labelled sample;
(e) assessing the first and second labelled samples for a plurality of oxidation states of the protein; and
(f) correlating the assessment from step (e) with the ROS associated pathology in the presence and absence of the intervention.

The intervention may be varied and includes administration of an agent intended to have a therapeutic effect on ROS associated pathology.

The method of the present invention may be conveniently performed using a kit comprising a series of reagents necessary to carry out the method. Thus, the present invention also provides a kit for assessing the oxidation states of a protein in a sample, the kit comprising:
(a) a first label adapted to selectively bind to a reduced cysteine group of the protein in the sample; and
(b) a reagent for selectively reducing at least one reversibly oxidised cysteine group of the protein in the sample.

Preferably, the kit further comprises a second label adapted to selectively bind to a reduced cysteine group of the protein formed by treatment with the reagent. Preferably, the first and second label are the same.

Preferably, the kit further comprises a substrate for receiving the sample, Preferably, the substrate comprises the first label, and is adapted to bind at least one reduced cysteine group of the protein from a whole blood sample. Preferably the substrate is an absorbent paper, such as filter paper. Preferably the substrate further comprises at least one sample identifier.

Preferably the kit further comprise a sample collection device. Preferably the sample collection device is adapted to enable collection of a capillary blood sample. Preferably the sample collection device is a skin pricking device. Preferably the sample collection device is a hand-held device adapted to enable collection of a capillary blood sample from a heel, finger or ear lobe of a patient. Preferably the sample collection device is a lancet.

Preferably the substrate is a dried blood spot card, such as a Perkin Elmar 226 Spot Saver Card. Preferably the first label is embedded in the dried blood spot card. Preferably the sample is a whole blood sample, such as a finger prick sample. Alternatively, the substrate comprises a separation membrane for separating one or more proteins in a sample from other whole blood components, such as red blood cells.

Preferably, the kit further comprises an extraction reagent adapted to extract at least a portion of the blood sample from the dried blood spot card. Preferably, the kit further comprises a protein isolation reagent adapted to separate the bound protein from the sample.

Preferably, the kit further comprises instructions to utilise the reagents therein according to the methods described herein.

General

Those skilled in the art will appreciate that the invention described herein is susceptible to variations and modifications other than those specifically described.

It is to be understood that the invention includes all such variations and modifications. The invention also includes all of the steps, features, compositions and compounds referred to or indicated in the specification, individually or collectively and any and all combinations or any two or more of the steps or features.

The present invention is not to be limited in scope by the specific embodiments or examples described herein, which are intended for the purpose of exemplification only. Functionally equivalent products, compositions and methods are clearly within the scope of the invention as described herein.

The entire disclosures of all publications (including patents, patent applications, journal articles, laboratory manuals, books, or other documents) cited herein are hereby incorporated by reference. No admission is made that any of the references constitute prior art or are part of the common general knowledge of those working in the field to which this invention relates.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

### EXAMPLES

The following methods serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### Example 1—Quantitative Assessment of Albumin Thiol Oxidation after Exercise

1. Materials/Methods

(a) Participants

Healthy adults, aged 18-32, participated, with ethics approved by the human ethics committee of The University of Western Australia.

(b) Materials

Double-deionized (DDI) water was used throughout. Protein molecular weight standards were purchased from Bio-Rad (Australia). Unless otherwise stated, all chemicals and reagents were obtained from Sigma-Aldrich (Castle Hill, Australia). Polyethylene glycol maleimide (malpeg), 5000 g/mol was purchased from JenKem Technology (USA).

(c) Blood Sample Preparation and Storage

For the analysis of albumin thiol oxidation, nine parts of blood was collected into a K₃EDTA tube (Minicollect tubes K₃EDTA; Greiner Bio-One, Austria), containing 1 part of the trapping solution made up of 62.5 mM malpeg, 40 mM imidazole and 154 mM NaCl diluted in DDI water, pH 7.4. Additional blood was collected into a second K₃EDTA tube without trapping solution for the analysis of total plasma albumin. The tubes were briefly vortexed and then centrifuged (3000 g, 10 min), and plasma collected. Plasma without trapping solution was immediately frozen in liquid nitrogen and stored at −80° C., whereas plasma with trapping solution was incubated at room temperature for 20 minutes and then frozen and stored.

For the identification of human albumin with and without malpeg on the gel, 0.9 mM commercial human serum albumin (HSA; Sigma) was prepared in SDS/Tris buffer, containing 0.5% (w/v) SDS and 0.5 mM Tris (pH 7.4). Nine parts of HSA sample was added to 1 part of a trapping solution made up of 62.5 mM polyethylene glycol maleimide (Malpeg, 5000 g/mol, JenKem Technology, USA), 40 mM imidazole and 154 mM NaCl diluted in DDI water, pH 7.4. Samples without the trapping solution was immediately frozen in liquid nitrogen and stored at −80° C., whereas plasma collected in the presence of the trapping solution (Malpeg) was incubated at room temperature for 30 minutes prior to being frozen and stored

(d) Sample Preparation

Plasma or HSA samples containing malpeg were thawed at 37° C. with agitation and then divided into two, 2.5 μl aliquots.

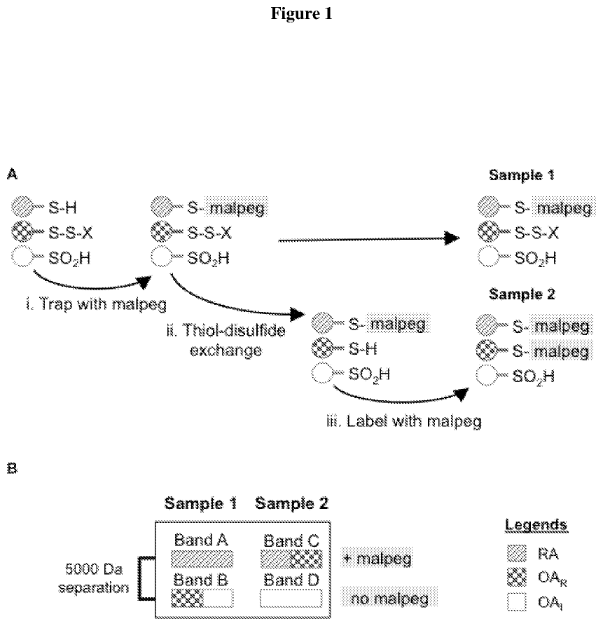

Procedure I involved adding SDS/Tris buffer (245 μl) containing 0.5% SDS and 0.5 mM Tris (pH 7.4) to aliquot 1 (Sample 1; FIG. 1a).

Procedure II involved adding 2.5 μl of 20 mM L-cysteine (pH 3) to aliquot 2, incubating for 30 min at room temperature, and then adding 5 μl of 25 mM malpeg with a further incubation for 15 minutes at room temperature. A sub-aliquot (4 μl) was added to 95 μl of SDS/Tris buffer (Sample 2; FIG. 1a).

(e) Gel Electrophoresis

Gels were hand casted using mini-protean plates (Bio-Rad). Briefly, the 16% resolving gel was made as per the Laemmli method [1]. For fluorescent imaging, 1% (v/v) of 2,2,2-trichloroethanol [2] was added. After the resolving gel was polymerised, the 4% stacking gel was poured on top of the resolving gel and after polymerization, the gels were stored in a dark cold room at least 3 hours before use.

Samples (5 μl of sample 1 and 5 μl sample 2) were mixed with equal parts of loading buffer containing 0.5M TRIS (pH 6.8), 3% (w/v) SDS, 30% (v/v) glycerol and 0.03% (w/v) bromophenol blue in DDI water. A 5 μl aliquot was loaded onto gels and gels were run at 250 V for 1 hr 45 mins in the cold and dark room. Following electrophoresis, the gel was washed twice with DDI water. The gel was placed on a UV transilluminator (ChemiDoc™, Biorad) for 5 min and then visualised with Image Lab™ software, Biorad. Images of the gels were analysed using NIH ImageJ software [Version 1.48v; [3]]. The image was inverted, and after background subtraction, and editing for speckling and noise a signal profile was plotted for each lane from the gel (Image J user guide 1.46r, 2012). The area under each peak were calculated using the trapezoid rule to give the intensity for each band [2].

(f) Protein Carbonyl Assays

Carbonyl groups formed on plasma albumin were determined with immunoblotting, which involved the processing of samples with positive and negative controls. Plasma samples were diluted 1:120 with 6% SDS. The positive control sample was incubated 1:1 with 50 mM HOCl for 1 hour, then diluted 1:60 with 6% (w/v) SDS. One part of diluted sample or positive control were added to one part of 10 mM dinitrophenyl hydrazine (10 mM DNPH/10% (w/v) TFA). The negative control sample was incubated with the same conditions of 10% (w/v) TFA, but without DNPH. After 15 minutes of incubation, one part of neutralization solution (30% glycerol/2 M Tris) was added to the DNPH and negative control treated samples. The treated samples were then diluted 10 times under reducing conditions, with 5 μl of treated samples separated by stain free SDS-PAGE (Biorad, 4-20% Mini-PROTEAN® TGX Siain-Free™ Precast Gels) and transferred (Biorad, Trans-Blot® Turbo™ Transfer System) onto nitrocellulose membrane (Biorad, Trans-Blot® Turbo™ Mini Nitrocellulose Transfer Packs) using conditions set at 2.5 A, up to 25 V for 10 minutes.

The membrane was subsequently washed in Tris-Buffered Saline Tween 20 (TBST) 5 times, for 3 minutes each (5×3 mins), and blocked with TBST/0.5% (w/v) non-fat dry milk. After one hour, the membrane was washed in TBST (5×3 mins), then incubated in polyclonal rabbit anti-DNP antibody (diluted 1:20000 in TBST/0.5% (w/v) non-fat dry milk). After an overnight incubation in the cold room, the membrane was washed in TBST (5×3 mins) and then treated with horseradish peroxidase-conjugated goat anti rabbit IgG (diluted 1:25000 in TBST/0.5% non-fat dry milk) for 1 hour at room temperature. A final wash with TBST (5×3 mins) was performed prior to visualisation of carbonylated albumin using ECL western blot detection reagent (Bio-rad, Clarity Western ECL substrate).

Albumin carbonylation was tabulated as a ratiometric value, using the carbonyl density divided by the amount of fluorescence signal of albumin from the stain free gel (loading control). That is: ratio=arbitrary amount of carbonylated albumin/arbitrary amount of albumin. The inter-assay coefficient of variation (standard deviation÷mean) for albumin carbonyl was 8.6% (n=9), similar to previous carbonyl methods with a coefficient of variations ranging from 7%-18% (Dayhoff-Brannigan et al. 2008 [5]; Matthaiou et al. 2014 [6]).

(i) Statistical Analysis

All data are presented as means±SE unless otherwise states. Means were compared using a t-test or one-way ANOVA with repeated measures where appropriate. Significance was accepted at p<0.05.

2. Results

(a) Method Development

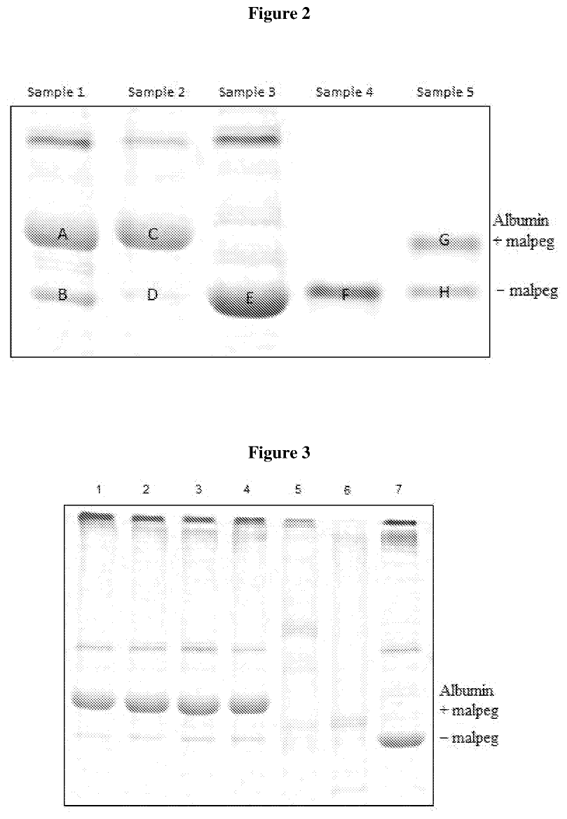

The quantitative analysis of plasma albumin thiol oxidation state involves maleimide labelling of cys-34 with malpeg, with labelled albumin then separated from unlabeled albumin by SDS-PAGE (FIGS. 1 & 2). In particular, Sample 1 was used for analysing the percentage of reduced (RA) and oxidised albumin (OA; FIG. 1b). Sample 2 was used to calculate the percentage of albumin in the reversibly oxidised form (OAR) and irreversibly oxidised form (OAI; FIG. 1b). For sample 1, the top band (A in FIG. 2) was reduced albumin (RA) and the bottom band (B in FIG. 2) was reversibly and irreversibly oxidised albumin (OAR & OAI). For sample 2, the top band (C in FIG. 2) was reduced (RA) and reversibly oxidised albumin (OAR) and the bottom band (D in FIG. 2) was irreversibly oxidised albumin (OAI). The percentage of albumin in the different forms was calculated as follows:
1. The percentage of reduced albumin (% RA)=band A/(band A+band B)*100
2. The percentage of irreversibly oxidised albumin (% OAI)=band D/(band D+band E)*100
3. The percentage of reversibly oxidised albumin (% OAR)=100−% RA−% OAT

The technique depends on labelling of the thiol of cys34 by malpeg. A concentration of 6.25 mM malpeg incubated for at least 15 minutes at room temperature was deemed sufficient for maximum labelling.

To measure reversibly oxidised cys34, a thiol-disulfide exchange reaction was used to generate a thiol on cys34 which could then be labelled with malpeg. Cysteine, reduced glutathione, N-acetylcysteine and mercaptoethanol were suitable thiol-disulfide exchange reagents, but dithiothreitol and TCEP were not (FIG. 3). Cysteine was used as a thiol-disulfide exchange reagent in all subsequent experiments. A cysteine concentration of at least 10 mM incubated for at least 15 minutes was sufficient to account for more maximal labelling of the reversibly oxidised thiol. After incubating with cysteine, incubating for at least 15 minutes with 12.5 mM of malpeg was sufficient for labelling of newly exposed thiol groups in cys34.

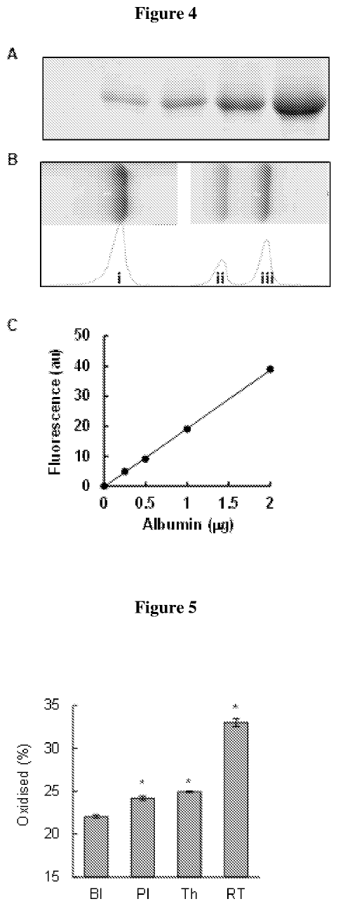

To quantify the relative oxidation of albumin fluorescent imaging of protein reacted with 2,2,2-trichloroethanol was used to achieve complete separation (FIG. 4bii, iii). There was a linear relationship between albumin content and gel band density (FIG. 4) for fluorescent imaging up to an albumin loading of 2 μg. This linear relationship meant that the relative fluorescent intensities for albumin bound and not bound to malpeg could be used to calculate relative oxidation. Consistent with this concept the fluorescent intensity of albumin with no malpeg added (22.5±0.7, arbitrary units, n=4) was comparable to the summed fluorescent intensity of sample 1 (22.7±0.5, arbitrary units, n=4) and sample 2 (22.5±0.6, arbitrary units, n=4) which contained both albumin bound and not bound to malpeg. The calculated oxidation of plasma albumin using the fluorescent imaging technique was reproducible, with a intra and inter assay coefficient of variation of 2.7% (n=12) and 4.7% (n=12) respectively.

(b) Collection and Preparation of Plasma Sample.

Protein thiol groups are sensitive to oxidation, so there is potential for artifactual oxidation during sample preparation. However, reacting the thiol group of cys34 with malpeg prevents oxidation. Three sample preparation techniques were tested, with malpeg added: to blood as soon as it was collected; to plasma following centrifugation; to freshly thawed plasma; and to plasma after 2.5 hours at room temperature. For all plasma samples, there was increased oxidation relative to the level of albumin oxidation in the blood sample to which malpeg had been added (FIG. 5).

(c) Comparison with Chromatography Technique

Chromatography based techniques have been used to measure fraction of albumin in the oxidised form. Using a chromatography technique described by Turell et al [4], bovine serum albumin samples were estimated to be 36±0.7% (n=5) oxidised, whereas the level of oxidation was estimated to be 42±0.1% (n=5) using the malpeg technique. Because of the discrepancy between the two measurements, bovine serum albumin samples were treated with hydrogen peroxide was used to completely oxidise thiol groups. Using the chromatography, albumin samples were 68±1.8% (n=5) oxidised, whereas the level of oxidation was estimated to be 98±0.1% (n=5) using the malpeg technique. These observations suggest the chromatography technique of Turell et al. underestimated the extent of albumin oxidation.

(d) Comparison of Albumin Oxidation Method with the Protein Carbonyl Assay

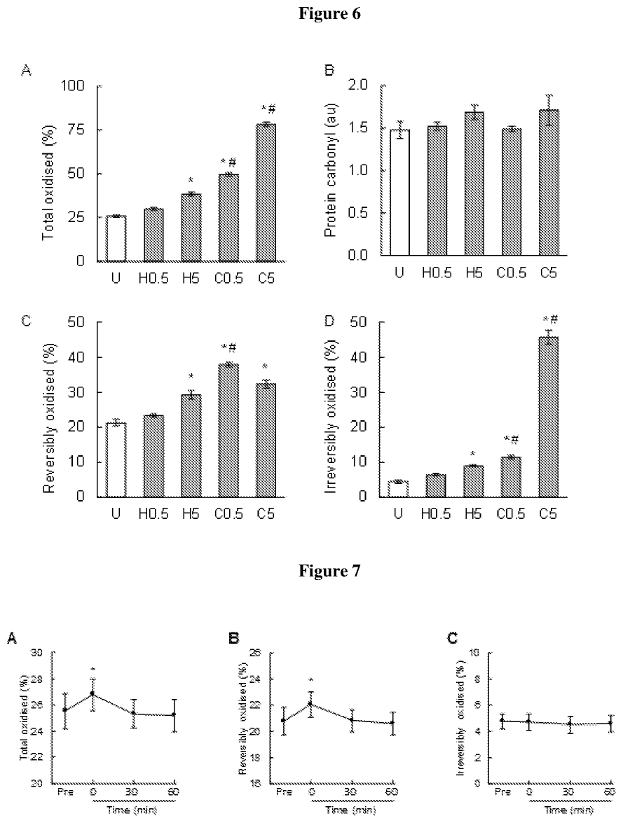

The sensitivity of the albumin oxidation method was compared to the protein carbonyl assay using two reactive oxygen species, hydrogen peroxide and hypochlorous acid. For hydrogen peroxide, concentrations of 0.5 mM and 5 mM caused significant increases in albumin Cys34 oxidation with no significant increases in protein carbonyl formation (FIG. 6). A similar pattern of oxidation was evident for hypochlorous acid, with a significant increase in albumin Cys34 oxidation, but no significant increase in protein carbonyl formation (FIGS. 6A & 6B). At equivalent concentrations, hypochlorous acid caused greater oxidation of albumin than hydrogen peroxide.

Both hydrogen peroxide and hypochlorous acid caused increases in reversibly and irreversibly oxidised albumin (FIG. 6C). However, hypochlorous acid at 5 mM caused a significantly lower increase in reversibly oxidised albumin than at 0.5 mM. This apparent discrepancy is addressed in the discussion.

(e) Applications: Quantitative Assessment of Human Albumin Thiol Oxidation after Exercise.

The sensitivity of the gel based method assay was tested by measuring human plasma albumin thiol oxidation after exercise. Participants performed a \dot{V}O_{2Peak} stationary cycling exercise test at an initial intensity of 50 watts, with the intensity increasing by 30 watts at 3 mins interval until volitional exhaustion or until the participant was unable to successfully maintain the required power output. Capillary blood samples were collected prior to and after exercise. Immediately after exercise, there was an increase in oxidised albumin which returned to pre-exercise levels by 30 mins post-exercise (FIG. 7). The increase in oxidised albumin was a consequence of an increase in reversibly oxidised albumin and not irreversibly oxidised albumin (FIG. 7).

As would be apparent, various alterations and equivalent forms of the examples may be provided without departing from the spirit and scope of the present invention. This includes modifications within the scope of the appended claims along with all modifications, alternative constructions and equivalents.

### Example 2—Protein Oxidation Levels in Individuals with Chronic Fatigue Syndrome

1. Materials/Methods

(a) Participants

Healthy volunteers (n=12) and people suffering from Chronic Fatigue Syndrome (n=12) performed 3 maximal intensity voluntary contractions involving a unilateral knee extension on an ergometer for 30 s, with 120 s of recovery between extensions. The knee angle was fixed at 70 degrees.

Blood samples were taken immediately before exercise, immediately after exercise, 15 min after exercise, and 30 min after exercise.

Participants were required to abstain from consuming alcohol, caffeine, and painkillers for 48 hours before testing. They were required to fast from 10 pm the day prior to testing. All testing began at 9 am.

(b) Materials

Double-deionized (DDI) water was used throughout. Protein molecular weight standards were purchased from Bio-Rad (Australia). Unless otherwise stated, all chemicals and reagents were obtained from Sigma-Aldrich (Castle Hill, Australia). Polyethylene glycol maleimide (malpeg), 5000 g/mol was purchased from JenKem Technology (USA).

(c) Blood Sample Preparation and Storage

For the analysis of albumin thiol oxidation, nine parts of blood was collected into a K₃EDTA tube (Minicollect tubes K₃EDTA; Greiner Bio-One, Austria), containing 1 part of the trapping solution made up of 62.5 mM malpeg, 40 mM imidazole and 154 mM NaCl diluted in DDI water, pH 7.4. Additional blood was collected into a second K₃EDTA tube without trapping solution for the analysis of total plasma albumin. The tubes were briefly vortexed and then centrifuged (3000 g, 10 min), and plasma collected. Plasma without trapping solution was immediately frozen in liquid nitrogen and stored at −80° C., whereas plasma with trapping solution was incubated at room temperature for 20 minutes and then frozen and stored.

For the identification of human albumin with and without malpeg on the gel, 0.9 mM commercial human serum albumin (HSA; Sigma) was prepared in SDS/Tris buffer, containing 0.5% (w/v) SDS and 0.5 mM Tris (pH 7.4). Nine parts of HSA sample was added to 1 part of a trapping solution made up of 62.5 mM polyethylene glycol maleimide (Malpeg, 5000 g/mol, JenKem Technology, USA), 40 mM imidazole and 154 mM NaCl diluted in DDI water, pH 7.4. Samples without the trapping solution was immediately frozen in liquid nitrogen and stored at −80° C., whereas plasma collected in the presence of the trapping solution (Malpeg) was incubated at room temperature for 30 minutes prior to being frozen and stored

(d) Sample Preparation

Plasma or HSA samples containing malpeg were thawed at 37° C. with agitation and then divided into two, 2.5 μl aliquots.

Procedure I involved adding SDS/Tris buffer (245 μl) containing 0.5% SDS and 0.5 mM Tris (pH 7.4) to aliquot 1 (Sample 1; FIG. 1a).

Procedure II involved adding 2.5 μl of 20 mM L-cysteine (pH 3) to aliquot 2, incubating for 30 min at room temperature, and then adding 5 μl of 25 mM malpeg with a further incubation for 15 minutes at room temperature. A sub-aliquot (4 μl) was added to 95 μl of SDS/Tris buffer (Sample 2; FIG. 1a).

(e) Gel Electrophoresis

Gels were hand casted using mini-protean plates (Bio-Rad). Briefly, the 16% resolving gel was made as per the Laemmli method [1]. For fluorescent imaging, 1% (v/v) of 2,2,2-trichloroethanol [2] was added. After the resolving gel was polymerised, the 4% stacking gel was poured on top of the resolving gel and after polymerization, the gels were stored in a dark cold room at least 3 hours before use.

Samples (5 μl of sample 1 and 5 μl sample 2) were mixed with equal parts of loading buffer containing 0.5M TRIS (pH 6.8), 3% (w/v) SDS, 30% (v/v) glycerol and 0.03% (w/v) bromophenol blue in DDI water. A 5 μl aliquot was loaded onto gels and gels were run at 250 V for 1 hr 45 mins in the cold and dark room. Following electrophoresis, the gel was washed twice with DDI water. The gel was placed on a UV transilluminator (ChemiDoc™, Biorad) for 5 min and then visualised with Image Lab software, Biorad. Images of the gels were analysed using NIH ImageJ software [Version 1.48v; [3]]. The image was inverted, and after background subtraction, and editing for speckling and noise a signal profile was plotted for each lane from the gel (Image J user guide 1.46r, 2012). The area under each peak were calculated using the trapezoid rule to give the intensity for each band [2].

2. Results

FIG. 16 shows the effect of isometric contraction on protein oxidation level in blood of individuals with CFS (n=12) and healthy sedentary individuals (n=12). Blood was collected pre-exercise (pre), and at 0, 15 and 30 minutes after exercise. Data are shown as mean, with error bars indicating standard errors of the mean. * indicates significant difference (p<0.05) between healthy and CFS participants. # indicates significance (p<0.05) from pre-exercise measurement.

The healthy participants experienced a post-contraction increase in the oxidation state of plasma albumin Cys34. A similar increase was not seen in participants with known Chronic Fatigue Syndrome. Results showed that there was an abnormal response to repeated isometric contraction in the blood of individuals with Chronic Fatigue Sydnrome.

### Example 3—Effect of Various Stressors on Reversibly Oxidized Albumin Levels

1. Materials/Methods

(a) Materials

Double-deionized (DDI) water was used throughout. Protein molecular weight standards were purchased from Bio-Rad (Australia). Unless otherwise stated, all chemicals and reagents were obtained from Sigma-Aldrich (Castle Hill, Australia). Polyethylene glycol maleimide (malpeg), 2000 g/mol was purchased from JenKem Technology (USA). Perkin Elmar 226 Protein save 5 Spot Cards were used.

(b) OxiMetric Dried Blood Spot Card Preparation Procedure

100 μL of 40 mM Imidazole was added to 12.5 mg of Methoxy polyethylene glycol in a 1.5 mL microfuge tube. The tube was vortexed for approximately 2 minutes. The resulting trapping agent was a clear solution with a final methoxy polyethelene glycol concentration of 62.5 mM.

5 μL of the prepared trapping agent was pippeted onto the center of each of the 5 spots on a blood card. The trapping agent spread out to cover approximately ¾ of the designated circle area of each of the blood spots. The blood card impregnated with trapping agent was placed into the supplied airtight container with desiccant, allowed to dry for at least 2 hours and stored in the same container until required for use.

(c) Blood Card and Finger Prick Sampling

A blood card comprising the trapping agent was removed from the desiccant container and place on a flat surface, with circles facing up. The container was resealed. A lancet was prepared, by removing the lance cap. The collection site was rubbed for approximately 20 seconds before lancing, The lancet was placed firmly against the puncture site, and the release button was pressed to puncture the skin. The puncture site was gently squeezed to produce a blood drop. One or two blood drops were applied to the center of a circle of the blood card. The sample was labelled with date, time and sample identifier. The top portion of the card was folded and tucked over the collected sample spots and the card was returned to the desiccant container.

The Blood Card can be stored at room temperature for several months, in the provided desiccant container. The desiccant container should be changed if the desiccant changes colour from orange to blue.

(d) Cibracron Blue Albumin Isolation Method and Thiol Oxidation Analysis

Blood Extraction from Blood Card

A 4.5 mm hole was punched through the centre of each spot on each the blood card. Each 4.5 mm blood card disk was placed into a separate well of a 96 well plate. 100 μl of 20 mM phosphate buffer, 0.05% Tween 20 (pH 7.1) was added to each well containing a blood card disk. The plate was incubated at room temperature on a plate mixer for 2 hours.

Reduction with Cysteine for Reversible Oxidation Analysis

A 40 μL aliquot was transferred from each well containing a blood card sample into a 0.5 mL microfuge tube. A 10 mM cysteine solution was prepared by mixing 3.5 mg of L-Cysteine hydrochloride with 100 μL of DDI in a 1.5 ml microfuge tube, which was gently vortexed for 30 seconds until dissolved, providing a solution with a cysteine concentration of 200 mM. The 200 mM solution was diluted (with DDI) 1:20 to give a final concentration of 10 mM Cysteine solution. 40 uL of the 10 mM Cysteine solution was added to the the 40 uL of blood card sample, and the sample was incubated for 30 minutes on a vortex at room temperature to allow for reduction of all thiols.

Labelling with Mal-PEG 2000 for Reversible/Irreversible Analysis

The samples were removed from the vortex, and 80 μL of a 12.5 mM Methoxy polyethylene glycol 2000 solution (12.5 mM Methoxy polyethylene glycol 2000 in 40 mM Imidazole pH 7.4) was added to each sample, and the samples were incubated for 30 minutes on a vortex at room temperature.

Albumin Isolation Using Cibacron Blue

5 μL aliquots of Cibacron blue were aliquoted into 0.5 mL microfuge tubes. 45 μL of 20 mM phosphate buffer was added, and gently mixed by flicking the tubes. The tubes were centrifuged for 1 minute, supernatant was removed and discarded. 40 μL of blood card disk solution and 160 μL of reduced blood card disk solution was added onto the Cibacron Blue, and gently mixed by flicking the tube. The tubes were incubated at room temperature for 10 minutes, and gently mixed by flicking the tubes. The tubes were centrigured for 1 minute, then the supernatant (containing un-bound whole proteins) was removed and discarded. 100 μL of 20 mM phosphate buffer was added to the Cibacron Blue-gel, and gently mixed before centrifuging then removing and discarding the supernatant to wash off any remaining unwanted whole blood components. The bound albumin was eluted by adding 25 μL of 1.4 M sodium chloride to the tube and gently mixing by flicking the tube. The tubes were centrifuged, and the supernatant was removed and stored in a 0.5 mL microfuge tube. The store supernatant samples contained relatively purified albumin.

Gel Electrophoresis and Thiol Analysis

Purified albumin solution was mixed with equal volumes of sample buffer (i.e. 20 μl albumin solution with 20 μL of sample buffer). The samples were vortexed, and then 20 μL samples were loaded onto a 16% polyacrylamide gel. The gel was run at 180 V, 70 mA for 2 hours. The gel was imaged on ChemiDoc MP Imaging system using 5-minute exposure. Image J was used to quantify ratio of oxidised albumin to total albumin. The ratio of oxidized albumin to total albumin=[intensity of oxidized band/(intensity of reduced band+oxidized band)]×100

(e) Statistical Analysis

All data are presented as means±SE unless otherwise states. Means were compared using a t-test or one-way ANOVA with repeated measures where appropriate. Significance was accepted at p<0.05.

2. Results

Effect of Exercise on Reversibly Oxidized Albumin Levels

A single participant performed a 5 km run. Exercise intensity was modified by increasing the running speed and doubled between moderate and high intensity. Blood samples were taken 24 hr after each run. A 2 day rest period was taken between the moderate and high intensity runs.

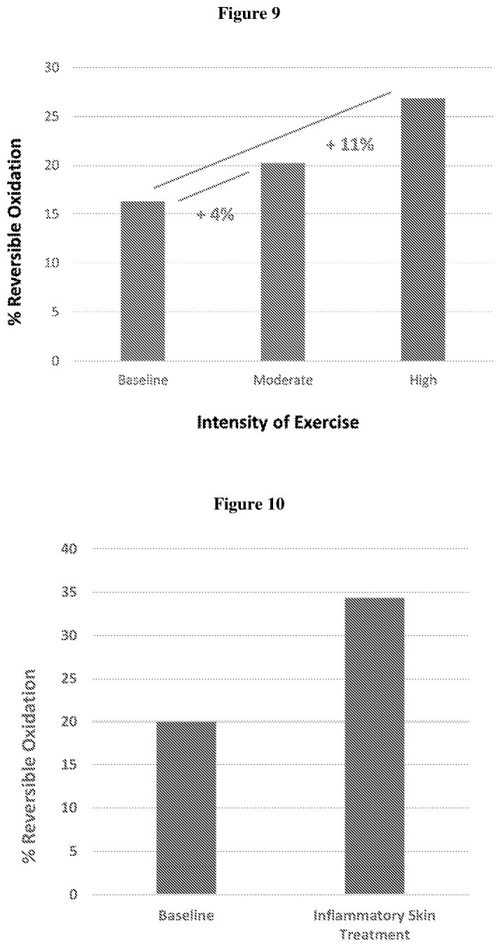

FIG. 9 shows increases in the amount of reversibly oxidized albumin detected in the moderate and high intensity exercise regimes compared to the baseline sample. FIG. 9 also demonstrates a correlation between the intensity of the exercise performed, and the amount of reversibly oxidized albumin present in the blood sample.

Effect of Inflammatory Skin Treatment on Reversibly Oxidized Albumin Levels

A female patient, aged 24, underwent medical microneedling treatment to the face. Blood samples were collected before treatment (baseline sample), and 24 hours after treatment was complete.

FIG. 10 shows a marked (15%) increase in reversibly oxidized albumin post treatment compared to the baseline sample.

Effect of Muscle Damage on Reversibly Oxidized Albumin Levels

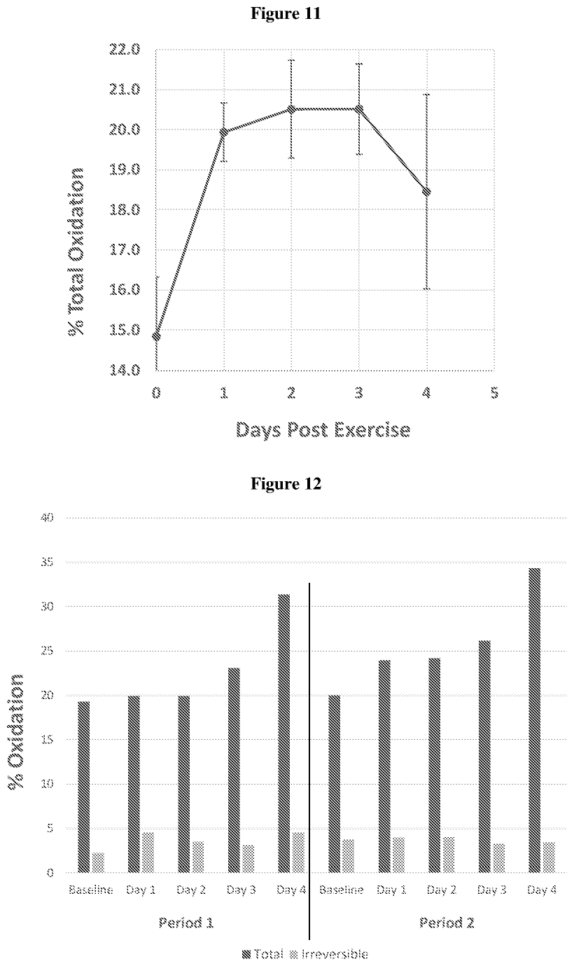

An untrained participant performed 4 sets of 6 repetitions (bicep curls) with a 5 kg weight. Blood samples were taken prior to performing the exercise and daily for 4 days post exercise. FIG. 11 shows that the patient samples demonstrated high oxidative stress, indicative of sustained muscle damage. The patients oxidative stress profile had not recovered to pre-exercise levels at day 4.

Reversible and Irreversible Oxidation Levels Following Exercise

A single participant performed two periods of a 4-day aerobic exercise trial. The two periods were separated by 2 weeks of rest. Exercise intensity (running duration and speed) were increased on day 2 and 4 with the highest intensity being on the 4^{th} day of each exercise period. All samples were obtained 24 hr after exercise.

FIG. 12 demonstrates large increases in the amount of reversibly oxidised albumin measured in samples taken after both moderate and high intensity exercise. There was minimal corresponding change observed to levels of irreversibly oxidized albumin after either moderate or high intensity exercise.

Oxidative Stress During Sickness

A single male subject who was believed to be suffering from acute sinusitis. Samples were taken in the morning approximately 24 hr apart.

FIG. 13 shows increases in both reversibly oxidized albumin and irreversibly oxidized albumin in pateints during periods of sickness.

Effect of Aerobic Exercise on Reversibly Oxidized Albumin Levels

Two patients with varying degrees of aerobic fitness undertook an exercise program. Patient 1 was categorised as having good aerobic fitness, and Patient 2 was categorized as having poor aerobic fitness. The exercise program consisted of a 5 km run, five twenty-minute soccer games, and a 100 m sprint. Samples were taken from the patients before the exercise program began and upon completion of the program.

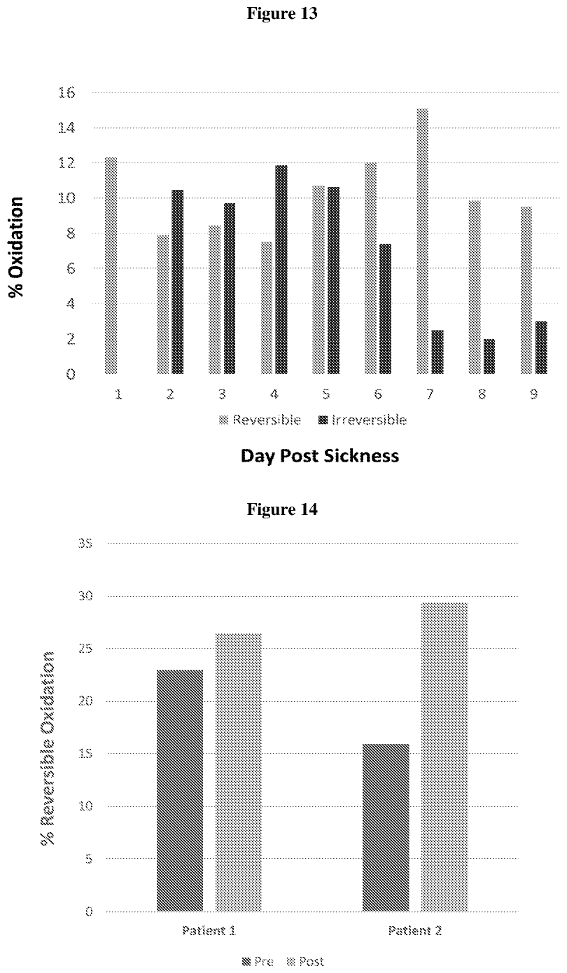

FIG. 14 shows minimal difference between the levels of reversibly oxidized albumin in Patient 1 pre and post exercise. There was a substantial increase in reversibly oxidized albumin in Patient 2 post exercise.

Reversibly Oxidized Albumin Levels During Muscle Injury Recovery

The Patient sustained a calf strain playing tennis. The patient complained that the muscle was very sore, and experienced significant loss of force. A physiotherapist confirmed a diagnosis of muscle injury and recommended a 1 to 2 week recovery period. Blood samples were taken from the patient regularly over a 10-day period beginning 24 hours post-injury.

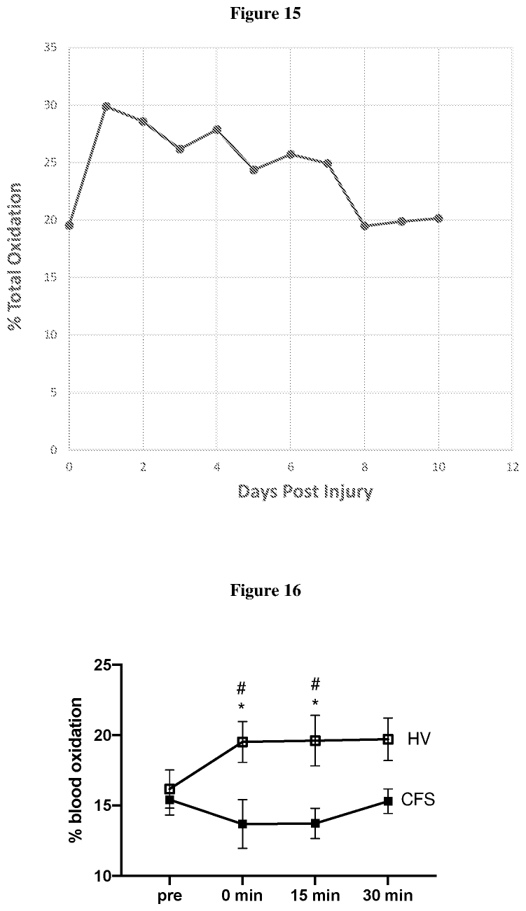

FIG. 15 shows a peak in the amount of reversibly oxidized albumin at day 2 post injury. A steady decrease in the amount of reversibly oxidized albumin was observed until day 10. The profile is consistent with the physiotherapists report and recommendation.

### Example 4—Method of Measuring Relative Oxidation Levels of a Protein Using Capillary Electrophoresis

1. Materials/Methods

(a) Materials

Double-deionized (DDI) water was used throughout. Protein molecular weight standards were purchased from Bio-Rad (Australia). Unless otherwise stated, all chemicals and reagents were obtained from Sigma-Aldrich (Castle Hill, Australia). Polyethylene glycol maleimide (malpeg), 5000 g/mol was purchased from JenKem Technology (USA).

(b) Blood Sample Preparation and Storage

For the analysis of albumin thiol oxidation, nine parts of blood was collected into a K₃EDTA tube (Minicollect tubes K₃EDTA; Greiner Bio-One, Austria), containing 1 part of the trapping solution made up of 62.5 mM malpeg, 40 mM imidazole and 154 mM NaCl diluted in DDI water, pH 7.4. Additional blood was collected into a second K₃EDTA tube without trapping solution for the analysis of total plasma albumin. The tubes were briefly vortexed and then centrifuged (3000 g, 10 min), and plasma collected. Plasma without trapping solution was immediately frozen in liquid nitrogen and stored at −80° C., whereas plasma with trapping solution was incubated at room temperature for 20 minutes and then frozen and stored.

(c) Plasma Sample Preparation and Storage

Trapped plasma samples containing malpeg were thawed at 37° C. with agitation and then divided into two 5 μl aliquots.

Samples were prepared using the following protocols.

Sample 1 (trapped)—5 μl of trapped-plasma (6.25 mM PEG) was diluted with 490 μl of SDS/Tris buffer). 0.5 uL of 100 uM cysteine (200 mM stock diluted ½ in DDI H20) was added. The sample is then placed on ice or stored at −80° C.

Sample 2 (trapped and reduced)—5 μI of trapped-plasma (6.25 mM PEG) was added to 5 μl of 20 mM L cysteine (200 mM stock diluted 1/10 in DDI H20). The sample was vortexed for 30 minutes to reduce reversibly oxidized albumin. 10 uL of 25 mM 10K PEG was then added and the sample was vortexed for 15 minutes to allow the PEG to bind to the albumin. 3 uL of 100 mM cysteine was added. Finally, 4.6 μl of the sample was diluted with 95 μl of SDS/Tris. The samples are then put on ice or stored at −80° C.

(e) Capillary Electrophoresis Under LabChip Protein Express Protocol

Samples were then loaded into LabChip GXII and run using the LabChip Protein Express protocol at an approximate albumin concentration of 0.023 mg/ml.

Results

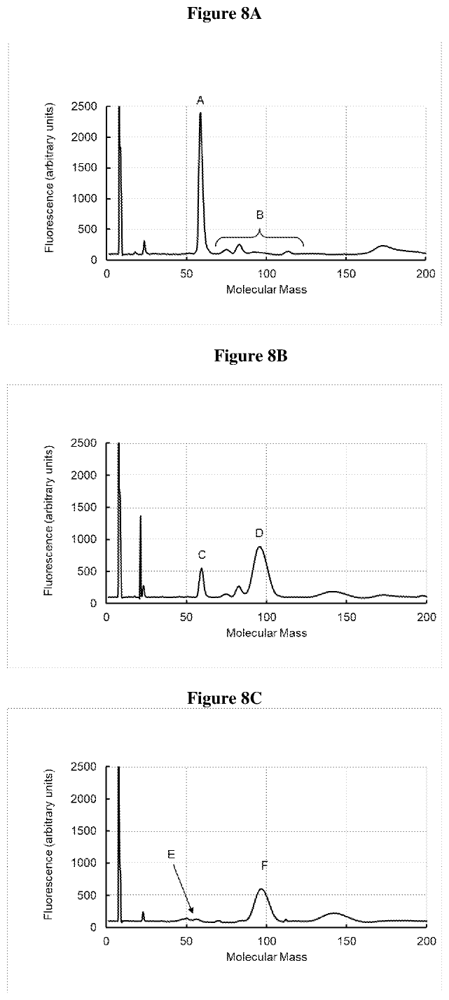

FIG. 8A shows total albumin (A) and other blood proteins (B) in an untreated plasma sample. FIG. 8B shows oxidised albumin (C) and reduced albumin (D) in a sample which was treated with malpeg. FIG. 8C shows irreversibly oxidised albumin (E) and reversibly oxidised and reduced albumin (F) in a sample that underwent a first malpeg treatment step, a reduction step (with cysteine) and a second malpeg treatment step.

### REFERENCES

[1] U. Laemmli, SDS-page Laemmli method, Nature 227 (1970) 680-5.
[2] C. L. Ladner, J. Yang, R. J. Turner, R. A. Edwards, Visible fluorescent detection of proteins in polyacrylamide gels without staining, Analytical biochemistry 326(1) (2004) 13-20.
[3] C. A. Schneider, W. S. Rasband, K. W. Eliceiri, NIH Image to Imagek 25 years of image analysis, Nature methods 9(7) (2012) 671-675.
[4] L. Turell, H. Botti, L. Bonilla, M. J. Tones, F. Schopfer, B. A. Freeman, L. Armas, A. Ricciardi, B. Alvarez, R. Radi, HPLC separation of human serum albumin isoforms based on their isoelectric points, Journal of Chromatography B 944 (2014) 144-151
[5] Dayhoff-Brannigan, M., Ferrucci, L., Sun, K., Fried, L. P., Walston, J., Varadhan, R., Guralnik, J. M. and Semba, R. D. (2008). “Oxidative protein damage is associated with elevated serum interleukin-6 levels among older moderately to severely disabled women living in the community.” The Journals of Gerontology Series A: Biological Sciences and Medical Sciences 63(2): 179-183.
[6] Matthaiou, C. M., Goutzourelas, N., Stagos, D., Sarafoglou, E., Jamurtas, A., Koulocheri, S. D., Haroutounian, S. A., Tsatsakis, A. M. and Kouretas, D. (2014). “Pomegranate juice consumption increases GSH levels and reduces lipid and protein oxidation in human blood.” Food and chemical toxicology 73: 1-6.

1-51. (canceled)

52. A method for assessing the oxidation states of a protein in a sample, the method comprising the steps of:
(a) contacting the sample with a first label adapted to selectively bind to a reduced cysteine group of the protein therein to form a first labelled sample;
(b) forming a sub-sample of the first labelled sample;
(c) treating the sub-sample to selectively reduce at least one reversibly oxidised cysteine group of the protein therein to form a treated sub-sample;
(d) contacting the treated sub-sample with a second label adapted to selectively bind to a reduced cysteine group of the protein therein formed during step (c) to form a second labelled sample; and
(e) assessing the first and second labelled samples for a plurality of oxidation states of the protein.

53. The method according to claim 52, wherein the oxidation states comprise a reversibly oxidised form.

54. The method according to claim 52, wherein the protein is a protein selected from the list comprising: albumin, alpha-2-macroglobulin, fibrinogen beta chain, haptoglobin, immunoglobulin lambda constant 2, inter-alpha-trypsin inhibitor heavy chain H2, serotransferrin, immunoglobulin gamma-1 heavy chain, fibrinogen gamma chain, and transthyretin.

55. The method according to claim 53, wherein the protein is a protein selected from the list comprising: albumin, alpha-2-macroglobulin, fibrinogen beta chain, haptoglobin, immunoglobulin lambda constant 2, inter-alpha-trypsin inhibitor heavy chain H2, serotransferrin, immunoglobulin gamma-1 heavy chain, fibrinogen gamma chain, and transthyretin.

56. The method according to claim 55, wherein the reversibly oxidised form of albumin comprises a reversibly oxidised cysteine group at cys34.

57. The method according to claim 52, wherein the first label is further adapted to trap the reduced cysteine group.

58. The method according to claim 52, wherein the first label is contacted with the sample less than 1 minute after the sample is taken.

59. The method according to claim 52, wherein the first label comprises a sulfhydryl-reactive chemical group.

60. The method according to claim 59, wherein the first label comprises a maleimide group; a haloacetyl group, such as an iodoacetyl or a bromoacetyl group; and/or a pyridyl disulphide group.

61. The method according to claim 52, wherein the first label is used at a concentration of at least 3 mM, 3.6 mM, 5 mM, 6 mM, 6.25 mM, 7 mM, 8 mM, 9 mM, or 10 mM

62. The method according to claim 52, wherein the first label is contacted with the sample for at least 5, 10, 15, or 20 minutes.

63. The method according to claim 52, wherein the first label further comprises a separation member adapted to facilitate separation of a labelled compound relative to unlabeled compounds.

64. The method according to claim 52, wherein the first label comprises a fluorescent compound.

65. The method according to claim 52, wherein the step of treating the sub-sample to selectively reduce at least one reversibly oxidised cysteine group of the protein therein comprises the step of contacting the sub-sample with an effective amount of a thiol containing agent.

66. The method according to claim 65, wherein the thiol containing agent is adapted to react with the reversibly oxidised cysteine group in a reaction with an equilibrium constant (K) value of between 1 and 2, 1 and 3, or 1 and 4.

67. The method according to claim 65, wherein the thiol containing agent is selected from the group comprising: cysteine, glutathione (reduced), mercaptoethanol, cysteamine, penicillamine, and N-acetylcysteine.

68. The method according to claim 65, wherein the thiol containing agent is used at a final concentration of at least 2 mM, 4 mM, 6 mM, 8 mM, 10 mM, 12 mM, 12.5 mM, 15 mM, or 20 mM.

69. The method according to claim 65, wherein the thiol containing agent is contacted with the subsample for at least 5, 10, 15, 20, or 30 minutes.

70. The method according to claim 52, wherein the second label is used at a concentration that is higher than that used for the first label.

71. The method according to claim 52, wherein the second label is contacted with the treated subsample for at least 5, 10, 15, or 20 minutes.

72. The method according to claim 52, further comprising the step of quantifying the amount of the identified oxidation states of the protein.
